(19) United States
(12) Patent Application Publication
(10) Pub. No.: US 2006/0052765 A1
(43) Pub. Date: Mar. 9, 2006

(54) Percutaneous lead for neurostimulation having a fluid delivery lumen

(76) Inventors: Stephen T. Pyles, Ocala, FL (US); Daniel A. Graubert, Etna, NH (US)

Correspondence Address: GARDNER GROFF, P.C., 2018 POWERS FERRY ROAD, SUITE 800, ATLANTA, GA, 30339, US

(21) Appl. No.: 11/217,061

(22) Filed: Aug. 31, 2005

Related U.S. Application Data
(60) Provisional application No. 60/606,172, filed on Aug. 31, 2004.

(51) Int. Cl. A61M 31/00

(52) U.S. Cl. 604/509

(57) ABSTRACT

A percutaneous lead with at least one electrode has a lumen or fluid canal extending the length of its biocompatible body. The lumen aids in accurately placing the percutaneous lead by allowing a practitioner to inject fluid, such as a saline solution, through the canal to displace fat, veins, adhesions, or connective tissues that prevent the passage or accurate placement of the lead in the epidural space. Optionally, an inflatable balloon is attached at the distal end of the percutaneous lead to further aid in displacing connective tissue.

### CROSS-REFERENCE TO RELATED APPLICATIONS

This application claims priority to U.S. Provisional Patent Application Ser. No. 60/606,172, filed Aug. 31, 2004, which is hereby incorporated herein by reference in its entirety for all purposes.

### TECHNICAL FIELD

The present invention relates generally to the field of surgical instruments and methods, and more particularly to a percutaneous lead having a fluid delivery lumen and a plurality of electrodes for neurostimulation.

### BACKGROUND OF THE INVENTION

Spinal cord stimulation is used to alleviate chronic pain by stimulating the central nervous system. Conventional percutaneous leads, such as the Medtronic PISCES-Quad® or Octad® Leads or the ANS Octrode® and Quattrode® Leads, are solid and have a plurality, but typically four or eight, electrodes. The percutaneous leads can be inserted through a needle and placed in the epidural space, in close proximity to the spinal cord. When activated, the electrodes deliver a precise, mild electrical impulse to the spinal cord or to a peripheral nerve. The electrical impulses activate pain inhibitory mechanisms to block the pain signal from reaching the brain.

However, accurately placing known electrodes can be rather difficult because the epidural space that surrounds the spinal cord typically contains fat, veins, adhesions, and connective tissue membranes which interfere with, and often prevent, the accurate placement of the electrodes.

Therefore, a need exists for an apparatus and method which would allow for greater ease in placing percutaneous electrodes in the epidural space.

### SUMMARY OF THE INVENTION

In an example form, the present invention is a percutaneous lead for placement in the epidural space of an animal or human subject. The lead has a biocompatible body portion defining an elongate shaft, at least a portion of which is flexible, at least one electrode positioned along the shaft, and a lumen extending through at least a portion of the shaft for carrying a fluid. The lead preferably further includes an outlet at a distal end of the shaft for discharging fluid through the lumen. Optionally, the lead includes an expandable, latex balloon in fluid communication with the lumen for displacing obstructions, such that the lumen can carry a sterilized fluid under sufficient pressure to expand the balloon. Also optionally, the lead further includes a second lumen for delivering a fluid directly to a tissue obstruction.

In another aspect, the present invention is a method of implanting a percutaneous lead in the spinal epidural space of a human or animal subject. The method includes the steps of inserting a percutaneous lead having a fluid delivery lumen with an outlet at a distal end thereof into the epidural space; injecting fluid through the percutaneous lead to displace tissue obstructions in the epidural space; and guiding the lead into a desired position in the epidural space. Preferably, the percutaneous lead has at least one electrode, and the method further includes delivery of therapeutic energy to tissue adjacent the electrode. Optionally, the step of injecting fluid comprises injecting through the outlet a fluid comprising saline, corticosteroid, and/or hyaluronidase into the area of the tissue obstruction. Also optionally, the method includes the steps of inflating and deflating a balloon positioned at an end of the lead to displace tissue obstructions within the epidural space; using fluoroscopy to guide placement of the percutaneous lead; and suturing the lead to anchor it in the desired position in the epidural space.

In yet another aspect, the present invention provides a kit for implanting a percutaneous lead. The kit preferably includes a needle, a sterile drape, a fluid coupling, a percutaneous lead having at least one electrode and fluid delivery lumen extending therethrough, suturing supplies, and/or various subcombinations thereof, within a case or other container.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a perspective view of a percutaneous lead having a fluid delivery lumen therethrough in accordance with an example embodiment of the present invention.

FIG. 2 shows a cross-sectional view of a portion of the percutaneous lead of FIG. 1.

FIG. 3 shows a perspective view of a percutaneous lead having a fluid delivery lumen extending therethrough, and a balloon for displacing connective tissue in accordance with another example embodiment of the present invention.

FIG. 4 shows placement of a percutaneous lead of FIG. 1 in the epidural space according to an example form of the invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

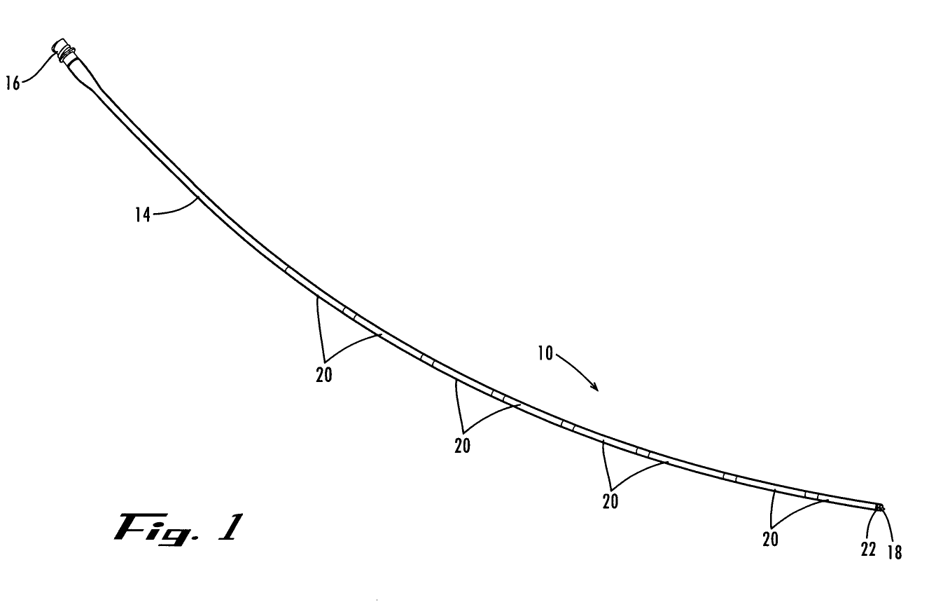

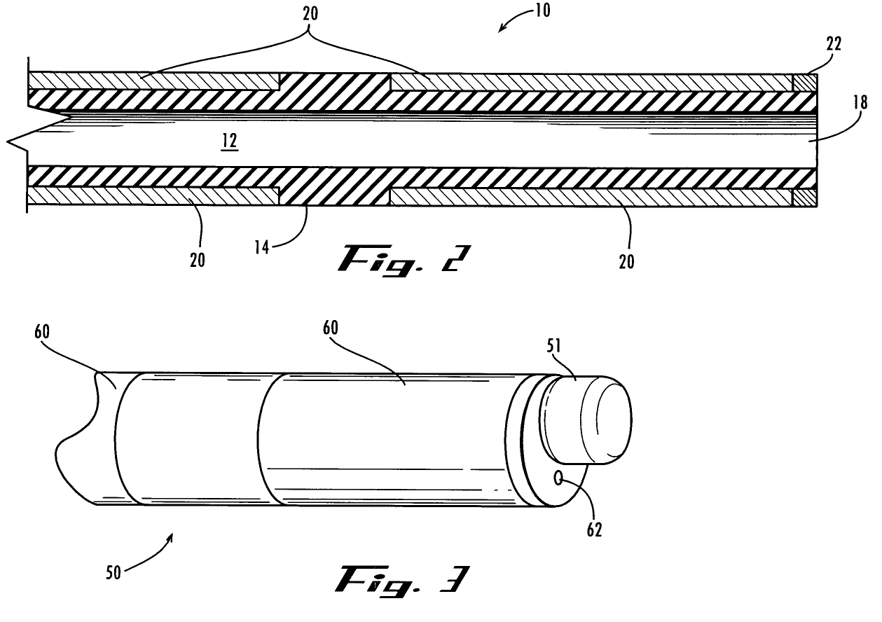

Referring to FIGS. 1 and 2, a percutaneous lead 10 having a fluid delivery lumen 12 extending therethrough is described by way of an example embodiment. Preferably, the percutaneous lead 10 has a biocompatible, somewhat flexible, electrically non-conductive, cylindrical body 14. Exemplary materials that can be used to construct the body include, but are not limited to, silicone, polyurethane, or polyethylene. Those skilled in the art will understand that various other biocompatible or biologically inert materials of construction can be used as well, without deviating from the scope of the present invention. The body 14 optionally includes a polyurethane insulation sheath for increased durability and longevity. The fluid delivery lumen or canal 12 preferably extends through or along substantially the entire length of the body 14 for carrying a fluid, such as a saline solution, from a fluid source directly to an area of a tissue obstruction or obstructive tissue within the subject's epidural space. As used herein, the terms tissue obstruction and obstructive tissue refer to any fat, vein, adhesion, connective tissue, or other obstruction in the epidural space that interferes with the proper placement of the percutaneous lead 10. Preferably, the lead 10 has a connector 16, such as a “leur-lock” type connector, at a proximal end thereof, for connecting to a fluid source to deliver fluid into the lumen 12. Those skilled in the art will understand that various other connectors for connecting the percutaneous lead 10 to the fluid source can be employed as well, without deviating from the scope of the present invention. At the distal tip (i.e., the end opposite the connector 16) is a discharge outlet 18 for discharging fluid from the lumen 12.

The lumen 12 preferably delivers pressurized fluid for direct injection into the area of the tissue obstruction. For example, a saline solution can be injected into the area of the tissue obstruction to help break up the obstruction. In one example embodiment, a mixture of saline, corticosteroid, and hyaluronidase is injected into the site of the tissue obstruction, via the fluid lumen 12 and the outlet 18, to reduce the inflammation. Preferably, the volume of the mixture is not more than about 20 milliliters. Also preferably, the amount of the corticosteroid administered is limited to about 20 milligrams to no more than about 80 milligrams, and the amount of the hyaluronidase is limited to about 150 USP units to no more than about 1500 USP units.

The percutaneous lead 10 includes at least one, and preferably, a plurality of electrodes 20 for spinal cord stimulation. Preferably, the plurality of electrodes 20 comprises four or eight cylindrical electrodes spaced along the length of the lead 10. One or more wires or other electrical conductors are preferably embedded in or on the body 14 to deliver electrical signals from an external source to the electrodes 20. FIGS. 1 and 2 show an example embodiment with eight such electrodes 20. In an example embodiment, the lead includes eight electrodes, each electrode being about 52 mm long, and the lead being about 60 cm long. In another embodiment, the lead 10 includes four electrodes 20, each electrode being about 24-34 mm long, and the lead being about 30 cm long. Those skilled in the art will understand how to configure the lead and to determine, for example, electrode material, size, shape, span, and spacing. Appropriate selection of the lead size and electrode configuration can be made in accordance with accepted medical protocol as determined by the treating physician.

Optionally, the percutaneous lead 10 includes a marker 22, such as a radiographic strip or band near the tip of the lead. The marker can aid the practitioner in guiding the lead 10 under fluoroscopy into a proper placement in the epidural space.

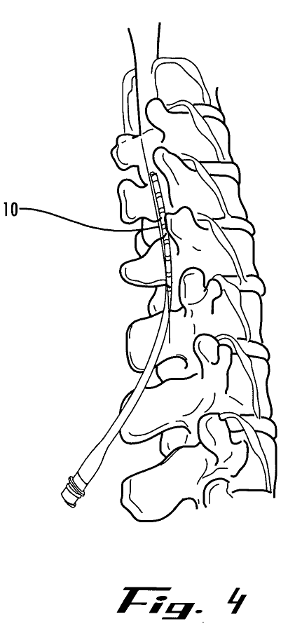

Another example embodiment of the present invention is shown in FIG. 3. The percutaneous lead 50 preferably comprises a plurality of electrodes 60, in substantially similar fashion to the lead 10 described above, and further comprises an inflatable and deflatable balloon 51. The balloon 51 is preferably connected at or near the distal end of the percutaneous lead 10, in fluid communication with the fluid lumen extending therethrough. The lumen delivers fluid from a remote fluid source, such as a sterilized liquid or air, under sufficient pressure to inflate and deflate the balloon 51. Preferably, the balloon 51 is constructed of a durable, yet distensible, material such as latex, although the present invention also contemplates the use of other distensible, biocompatible materials. The practitioner can alternately inflate and deflate the balloon 51 to displace tissues that prevent the passage or placement of the percutaneous lead. Optionally, the balloon 51 is detachable and retractable through the lumen, so that once the percutaneous lead is properly placed, the practitioner can disengage the balloon from the lead and remove it, with, for example, a stylet or some other device. Optionally, the lead 50 includes a second fluid delivery lumen 62 extending therethrough. In this embodiment, the first lumen is used to deliver fluid directly to the area of the tissue obstruction, while the second lumen is used to deliver fluid to distend the balloon.

Optionally, the leads 10 and 50 can be steerable, as with a stylet or other device. Also optionally, a fiberoptic scope could be inserted through the lumen for visualization of internal tissue.

In a preferred manner of use, a guide needle is positioned generally in the epidural space of a human or animal subject. The percutaneous lead is connected to a fluid source and is inserted through the guide needle into the epidural space. Preferably, the practitioner uses fluoroscopy to guide the placement of the guide needle and/or the lead. As the practitioner is guiding the lead into the desired location, the practitioner can inject fluid from the fluid source through the lumen into the epidural space to displace tissue obstructions such as fat, veins, adhesions, and connective tissue membranes, which would otherwise interfere with the accurate placement of the electrodes. Optionally, if an embodiment including a distensible balloon is utilized, the practitioner directs fluid delivery to expand and contract the balloon for displacement of obstructions. Once the lead is positioned as desired in the epidural space of the patient, for example as seen in FIG. 4, the lead can be secured in place with sutures. The lead is disconnected from the fluid source and is connected to a power source for delivery of electrical energy to the electrode(s). The power source may be external, or may be implanted internally, for example in the patient's abdomen or elsewhere. An internal or external controller is preferably used to control the internal power source and activate the electrodes according to a physician prescribed treatment regimen. The percutaneous lead 10 thus functions both as a typical catheter when implanting the lead and as a spinal cord stimulator once implanted.

Optionally, the tools and supplies that the practitioner uses to implant the lead of the present invention into the patient are assembled into a self-contained kit. For example, the kit includes a guide needle, a lead, a sterile drape, a power source, a fluid coupling, and suturing supplies, or any subcombination thereof, within a case or other container.

While the invention has been described with reference to preferred and example embodiments, it will be understood by those skilled in the art that a variety of modifications, additions and deletions are within the scope of the invention, as defined by the following claims.

What is claimed is:

1. A percutaneous lead for placement in the epidural space of a human or animal subject, the lead comprising:
a biocompatible body portion defining an elongate shaft, at least a portion of which is flexible;
at least one electrode positioned along the shaft; and
a lumen extending through at least a portion of the shaft for carrying a fluid.

2. The percutaneous lead of claim 1, further comprising an outlet at a distal end of the shaft for discharging fluid delivered through the lumen.

3. The percutaneous lead of claim 1, further comprising an inflatable balloon in fluid communication with the lumen.

4. The percutaneous lead of claim 3, wherein the lumen carries a sterilized fluid under sufficient pressure to expand the balloon.

5. The percutaneous lead of claim 3, wherein the balloon is formed of latex.

6. The percutaneous lead of claim 3, further comprising a second lumen for discharging a fluid directly to a tissue obstruction.

7. The percutaneous lead of claim 1, further comprising a leur-lock connector at a proximal end of the shaft for connecting a fluid source into communication with the lumen.

8. The percutaneous lead of claim 7, wherein the fluid source delivers a pressurized saline solution.

9. The percutaneous lead of claim 1, further comprising a radiographic marker on the shaft, for observation of the lead under fluoroscopy.

10. A method of implanting a percutaneous lead in the epidural space, comprising:
inserting a percutaneous lead having a fluid delivery lumen with an outlet at a distal end thereof into the epidural space;
injecting fluid through the percutaneous lead to displace tissue obstructions in the epidural space; and
guiding the lead into a desired position in the epidural space.

11. The method of claim 10, wherein the percutaneous lead comprises at least one electrode, said method further comprises delivery of therapeutic energy to tissue adjacent the electrode.

12. The method of claim 10, further comprising the step of inflating and deflating a balloon positioned at an end of the lead to displace tissue obstructions.

13. The method of claim 10, further comprising the step of using fluoroscopy to guide placement of the percutaneous lead.

14. The method of claim 10, wherein the step of injecting fluid comprises discharging through the outlet a fluid comprising saline, corticosteroid, and/or hyaluronidase into the area of the tissue obstruction.

15. The method of claim 10, further comprising suturing the lead in the desired position in the epidural space.

16. A kit, comprising:
a needle;
a sterile drape;
a fluid coupling;
a percutaneous lead having at least one electrode and a fluid delivery lumen extending therethrough; and
suturing supplies.
